(19) United States
(12) Patent Application Publication
(10) Pub. No.: US 2022/0142938 A1
(43) Pub. Date: May 12, 2022

(54) PLANT-DERIVED EXTRACELLULAR VESICLE (EVS) COMPOSITIONS AND USES THEREOF

(71) Applicant: EVOBIOTECH S.R.L., Torino (IT)

(72) Inventors: Giovanni CAMUSSI, Torino (IT); Chiara GAI, Bagnasco (Cuneo) (IT); Margherita Alba Carlotta POMATTO, Piossasco (Torino) (IT)

(21) Appl. No.: 17/438,020

(22) PCT Filed: Mar. 12, 2020
(86) PCT No.: PCT/EP2020/056632

§ 371 Date: Sep. 10, 2021

(30) Foreign Application Priority Data
Mar. 13, 2019 (IT) 102019000003639

(51) Int. Cl. A61K 9/51; A61K 36/00

(52) U.S. Cl. CPC A61K 9/5184; A61K 36/00

(57) ABSTRACT

A composition comprising a population of plant-derived extracellular vesicles (EVs) having a diameter ranging from 10 to 500 nm and showing pro-angiogenic, and anti-bacterial activity, for use in therapeutic applications is provided. A method for loading one or more negatively-charged biologically-active molecules into the population of plant-derived extracellular vesicles (EVs) is also provided.

### TECHNICAL FIELD

The present invention relates to plant-derived extracellular vesicle (EVs) compositions and their therapeutic applications.

### BACKGROUND

Extracellular vesicles (EVs) are a heterogeneous population of particles released by virtually all living cells. They have been purified from nearly all mammalian cell types and body fluids, as well as from lower eukaryotes, prokaryotes and plants. They mainly include microvesicles, released through the budding of the plasma membrane, and exosomes, derived from the endosomal compartment. Extracellular vesicles are referred to as “particles”, “microparticles”, “nanovesicles”, “microvesicles” and “exosomes”. [Yáñlez-Mó M, et al. Biological properties of extracellular vesicles and their physiological functions. J Extracell Vesicles. 2015 May 14; 4:27066 doi: 10.3402/jev.v4.27066; Lötvall J, et al. Minimal experimental requirements for definition of extracellular vesicles and their functions: a position statement from the International Society for Extracellular Vesicles. J Extracell Vesicles. 2014 Dec. 22; 3:26913. doi: 10.3402/jev.v3.26913.; Harrison P, et al. Extracellular Vesicles in Health and Disease. CRC Press, pages 1-5, 2014].

EVs contain a complex and variable cargo of cytoplasmic proteins, surface receptors, certain lipid-interacting proteins, DNA and RNA molecules. By transferring their cargo, EVs play a key role as mediators of intercellular communication.

Edible plant-derived EVs in their native form, not loaded with exogenous molecules, will be herein referred to as “native EVs”.

Native EVs are known to be effective for the treatment of leukemia [WO2016166716A1] and colitis [Ju S, et al. Grape exosome-like nanoparticles induce intestinal stem cells and protect mice from DSS-induced colitis. Mol Ther. 2013 July; 21(7):1345-57. doi: 10.1038/mt.2013.64.] by oral administration.

Native nanovesicles derived from grapes, grapefruit, ginger and carrots have shown anti-inflammatory effects in chronic inflammatory bowel disease [Zhang M, et al. Edible ginger-derived nanoparticles: A novel therapeutic approach for the prevention and treatment of inflammatory bowel disease and colitis-associated cancer. Biomaterials. 2016 September; 101:321-40. doi:10.1016/j.biomaterials.2016.06.018; Ju S, et al. Grape exosome-like nanoparticles induce intestinal stem cells and protect mice from DSS-induced colitis. Mol Ther. 2013 July; 21(7):1345-57. doi: 10.1038/mt.2013.64].

WO2017/052267 discloses the use of topically administered edible native plant-derived EV to promote skin improvement in terms of wrinkle formation, moisturization, whitening, epithelial cell proliferation and collagen deposition.

To the inventors' knowledge, the prior art does not disclose plant-derived EVs effects on angiogenesis and bacteria viability when administered topically on wound and skin lesions characterized by ischemia and impaired angiogenesis, or increased exposition to bacterial infection.

Since EVs naturally protect and transfer their cargo to target cells, they represent a useful alternative to synthetic and exogenous particles, such as liposomes, cationic nanoparticles, EV-mimetic nanovesicles and polypeptide-based vesicles to convey therapeutic agents. EVs can exploit their natural mechanism of action and overcome some of the limitations of assembled-particles, including immunogenicity, toxicity, administration of exogenous particles, limited cell uptake and chemical assemblage of particles.

In recent years, numerous techniques have been investigated to transfer different molecules (RNAs, DNAs, drugs) into EVs. EV-associated nucleic acids are protected from degrading enzymes present in the microenvironment and could be delivered to target cells. Methods aimed to introduce molecules into EVs include electroporation, sonication, transfection, incubation, cell extrusion, saponin-mediated permeabilization, and freeze-thawing.

WO2017/004526A1 discloses the use of microvesicles derived from grape, grapefruit as carriers for miR18a and miR17 to be used as anticancer drugs, or for tracers to be used for diagnosis.

In order to overcome the limitations and drawbacks of the prior art, the present invention provides a composition comprising a population of plant-derived extracellular vesicles (EVs) as well as a method for loading one or more biologically active molecule into the population of plant-derived extracellular vesicles (EVs), as defined in the appended independent claims. The dependent claims identify further advantageous features of the claimed composition and method. The subject-matter of the appended claims forms an integral part of the present description.

### DETAILED DESCRIPTION OF THE INVENTION

The present invention relates to a composition comprising a population of plant-derived extracellular vesicles (EVs), wherein the plant-derived extracellular vesicles (EVs) in said population are enclosed or delimited by a lipid bilayer membrane and are characterized in that they have a diameter of from 10 to 500 nm, a protein content in the range of from 1 to 55 ng/109 EVs, an RNA content in the range of from 10 to 60 ng/1010 EVs, and which further characterized in that they show pro-angiogenic and anti-bacterial activity, for use in the therapeutic treatment of a disease or condition selected from the group consisting of ulcers, dermatites, corneal damages, eye diseases, mucosal lesions and infective lesions.

As used herein, the term “plant-derived extracellular vesicles” or “plant-derived EVs” refers to nanoparticles derived from plant cells, which are delimited or encapsulated by a phospholipid bilayer and which carry lipids, proteins, nucleic acids and other molecules derived from the cell they are derived from. Conventionally, the extracellular vesicles have a diameter in a range of 10-1000 nm.

The present invention makes use of a selected population of plant-derived extracellular vesicles (EVs) which have a diameter in the range of from 10 to 500 nm, preferably in the range of from 20 to 400 nm, even more preferably in the range of from 25 to 350 nm. The plant-derived extracellular vesicles used in the present invention may be native EVs or engineered EVs as illustrated in the following examples.

The expressions “protein content” and “RNA content” encompasses both the internal and the membrane content of the EVs used in the present invention.

Examples of the lipids in the EVs used in the present invention comprise, but are not limited to, 24-Propylidene cholesterol, Beta sitosterol, Campesterol, Dipalmitin, Eicosanol and/or Glycidol stearate.

In a still further preferred embodiment of the invention, the EVs population is derived from a plant selected from the group consisting of: the family Rutaceae, such as the genus Citrus; the family Rosaceae, such as Malus pumila; the family Vitaceae, such as Vitis vinifera; the family Brassicaceae, such as Anastatica hierochuntica; the family Selaginellaceae, such as Selaginella lepidophylla; the family Asteraceae, such as Calendula officinalis; the family Oleaceae, such as Olea europaea; the family Xanthorrhoeaceae, such as Aloe vera; the family Nelumbonaceae, such as Nelumbo; the family Araliaceae, such as subgenus Panax; the family Lamiaceae, such as Lavandula; the family Hypericaceae, such as Hypericum perforatum; the family Pedaliaceae, such as Harpagophytum procumbens; the family Ginkgoaceae, such as Ginkgo biloba; the family Piperaceae, such as Piper kadsura or Piper futokadsura; the family Rubiaceae, such as Hedyotis diffusa. Non-limiting examples of plants of the genus Citrus are lemon, orange, tangerine, clementine, bergamot, pompia.

The scope of the invention includes both compositions containing EVs derived from a single plant species and compositions containing EVs derived from a plurality of plant species.

The EVs used in the present invention are characterized in that they show pro-angiogenic, and anti-bacterial activity.

Within the present description, the expression “pro-angiogenic effect” is intended as the promotion of endothelial cells proliferation or vessel formation by endothelial cells and increased release of pro-angiogenic mediators in vitro or in vivo. Angiogenesis is a fundamental biologic process and its impairment is involved in the pathogenesis of several diseases, including ischemic ulcers, such as pressure ulcers, arterial ulcers, venous ulcers, diabetic ulcers, ischemic ulcers, exudative ulcers, dysmetabolic ulcers, traumatic ulcers, burns, fistulae, psoriasis, keratosis, keratitis, burns, fistulae, fissures, mucosal lesions (such as traumatic lesions due to prothesis and such, diabetic, mouth, decubital, genital mucosal lesions), corneal damages/eye diseases (including ulcers, traumatic injuries, degeneration injuries, abrasions, chemical injuries, contact lens problems, ultraviolet injuries, keratitis), dry eye, conjunctivitis, dermatitis (including acne, eczema, seborrheic dermatitis, atopic dermatitis, contact dermatitis, dyshidrotic eczema, neurodermatitis, dermatitis herpetiformis), androgenic alopecia, pruritus, cellular damage induced by pro-apoptotic drugs aimed to treat pre-cancerous lesions (e.g. actinic keratosis). Accordingly, the EVs used in the present invention are particularly useful in the treatment of such diseases. The native plant-derived extracellular vesicles with pro-angiogenic effect used in the present invention are preferably derived from Citrus plants: lemon, orange, tangerine, clementine, bergamot, pompia; from Rutaceae family, such as Citrus; from Rosaceae family, such as Malus pumila; from Vitaceae family, such as Vitis vinifera; from Brassicaceae family, such as Anastatica hierochuntica; from Selaginella lepidophylla; from Asteraceae family, such as Calendula officinalis; from Oleaceae family, such as Olea europaea; from Xanthorrhoeaceae family, such as Aloe vera, from Nelumbonaceae family, such as Nelumbo; from Araliaceae family, such as subgenus Panax; from Lamiaceae family, such as Lavandula; from Hypericaceae family, such as Hypericum perforatum; from Pedaliaceae family, such as Harpagophytum procumbens; from Ginkgoaceae family, such as Ginkgo biloba; Piperaceae family, such as Piper kadsura or Piper futokadsura; Rubiaceae family, such as Hedyotis diffusa.

Within the present description, the expression “anti-microbial effect” is intended to indicate any effect able to kill microbes, microbicidal, or able to inhibit bacterial growth, biostatic. Bacterial infections are common and cause diseases and wound complications, including in mucosal lesions (such as traumatic lesions due to prothesis and such, diabetic, mouth, decubital, genital mucosal lesions), infective lesions (such as virus infections, herpes infections, bacterial infections), ulcers (including diabetic, arterial, venous, dysmetabolic, exudative, ischemic, pressure), burns, fistulae, corneal damages/eye diseases (including ulcers, traumatic injuries, degeneration injuries, abrasions, chemical injuries, contact lens problems, ultraviolet injuries, keratitis), dry eye, conjunctivitis, dermatitis (including acne, eczema, seborrheic dermatitis, atopic dermatitis, contact dermatitis, dyshidrotic eczema, neurodermatitis, dermatitis herpetiformis), traumatic ulcers, cellular damage induced by pro-apoptotic drugs aimed to treat pre-cancerous lesions (such as actinic keratosis). Accordingly, the EVs used in the present invention are particularly useful in the treatment of such diseases.

The plant-derived extracellular vesicles with anti-microbial effect used in the present invention are preferably derived from citrus plants: lemon, orange, tangerine, clementine, bergamot, pompia; from Rutaceae family, such as Citrus; from Rosaceae family, such as Malus pumila; from Vitaceae family, such as Vitis vinifera; from Brassicaceae family, such as Anastatica hierochuntica; from Selaginella lepidophylla; from Asteraceae family, such as Calendula officinalis; from Oleaceae family, such as Olea europaea; from Xanthorrhoeaceae family, such as Aloe vera, from Nelumbonaceae family, such as Nelumbo; from Araliaceae family, such as subgenus Panax; from Lamiaceae family, such as Lavandula; from Hypericaceae family, such as Hypericum perforatum; from Pedaliaceae family, such as Harpagophytum procumbens; from Ginkgoaceae family, such as Ginkgo biloba; Piperaceae family, such as Piper kadsura or Piper futokadsura; Rubiaceae family, such as Hedyotis diffusa.

As mentioned above, the scope of the invention also includes a method for loading one or more negatively-charged biologically active molecules into a population of plant-derived extracellular vesicles (EVs) as defined above. The resulting EVs, loaded with one or more negatively-charged biologically-active molecules, shall be referred herein below as “loaded EVs”.

The method of the invention is based on bridge formation by means of a polycationic substance between the negatively charged EVs and the negatively charged biologically active molecules. The expression “negatively-charged biologically-active molecules” includes, but is not limited to, drugs, nucleic acid molecules, and liposoluble molecules such as liposoluble vitamins. Nucleic acid molecules include, but are not limited to, DNA and RNA molecules, including e.g. miRNA, mRNA, tRNA, rRNA, siRNA, regulating RNA, non-coding and coding RNA, DNA fragments, DNA plasmids). The loaded EVs, resulting from the method of the invention, are capable of protecting the loaded biologically active molecules from degradation and to transfer them to target cells. The loaded biologically active molecules preferably have a therapeutic potential.

The method of the invention comprises contacting the population of plant-derived extracellular vesicles (EVs) as defined above with a polycationic substance and the negatively-charged biologically active molecule and co-incubating. After co-incubation, the EVs are purified from the polycationic substance and the remaining free negatively-charged active molecules.

In a first embodiment, the EVs are first contacted and co-incubated with the polycationic substance to allow binding of the polycationic substance to the surface of the EVs and then the mixture of EVs and polycationic substance is contacted and co-incubated with the negatively-charged active molecules.

In a second embodiment, the polycationic substance and the negatively-charged active molecules are mixed together and then added to the EVs.

According to a preferred embodiment, the polycationic substance is selected from the group consisting of protamine, polylisine, cationic dextrans, salts thereof and combinations thereof. A preferred protamine salt is protamine hydrochloride.

As mentioned above, after loading the EVs are purified. Suitable purification techniques include, but are not limited to, gradient ultracentrifugation, ultrafiltration, diafiltration, tangential flow filtration, precipitation-based methods, chromatography-based methods, concentration, immunoaffinity capture-based techniques and microfluidics-based isolation techniques.

As it will be illustrated in the following experimental section, the inventors loaded EVs with synthetic miRNA molecules, then verified by qRT-PCR analysis that the miRNA molecules had been incorporated into the EVs. By qRT-PCR analysis and confocal microscopy, the inventors also verified that the miRNA-loaded EVs were capable of efficiently transfer their cargo to target cells. The use of mammalian miRNA not present in vegetables allows an efficient evaluation of loading. Moreover, miRNAs transferred to target cells were shown to be biologically active and to affect the expression of target mRNAs in cells.

As mentioned above, the loaded EVs resulting from the method of the present invention can be used to vehicle several negatively-charged biologically active molecules through EVs. For instance, miRNAs are involved in different important key pathways in both physiological and pathological processes. Some miRNAs are e.g. reported in the scientific literature to be essentially involved in cancer angiogenesis and regenerative processes. As a demonstration of the efficacy of the method, EVs were efficiently loaded with pro-regenerative miRNAs, such as miR-21 and miR-126, and with anti-angiogenic and anti-tumor miRNAs or miRNA inhibitors. The loaded EVs showed and enhanced efficacy as compared to the native EVs.

Accordingly, the method of the present invention can be used to produce loaded EVs with enhanced therapeutic effects, including pro-angiogenic and anti-bacterial properties, or to add new therapeutic activities to native EVs for pro-regenerative purposes, which include, but are not limited to, anti-angiogenic effects.

Alternatively, the method of the present invention can be used to produce loaded EVs with specifically modulated biological effects, for example an abolished proangiogenic effect, without affecting the anti-bacterial properties and vice versa.

The method of the present invention can also be used to modulate the intrinsic biological effects of EVs in order to obtain loaded EVs with custom-tailored selected and specifically required biological activity.

The method of the present invention can be used to produce loaded EVs that contain one or more exogenous molecules or loaded EVs enriched with biologically active endogenous compounds. Optionally, the method of the present invention can be used to improve the efficacy of EV-loading using any protocol aimed to introduce molecules inside EVs, including electroporation, sonication, transfection, incubation, cell extrusion, saponin-mediated permeabilization, and freeze-thaw cycles. As an example, the inventors showed that protamine-based EV loading associated with electroporation is capable of increasing loading.

The method of the present invention can also be used in combination to the loading of plant-derived EVs loaded with liposoluble molecules.

Accordingly, the present invention encompasses loading of plant-derived EVs to potentiate their native effect on cellular regeneration. The beneficial effect of plant-derived EVs can be enhanced by loading liposoluble molecules, such as anti-oxidant vitamins. Liposoluble molecules, in their native or modified form, are effectively incorporated into the EVs. Thus, plant-derived EVs can be loaded with antioxidant molecules, such as A and E vitamins, to enhance their beneficial effects.

Native and loaded EVs are administrable in several ways depending on the target site. For cutaneous and external mucosal repair, EVs can be administered topically, whereas oral administration is preferred to reach the digestive system.

Accordingly, the composition of the present invention, which comprises the population of plant-derived EVs as defined above, wherein the EVs are either native or loaded with exogenous or endogenous negatively-charged biologically active molecules, can be provided as a pharmaceutical composition formulated e.g. for topic application, local injection or oral administration, or can be provided as a food supplement preparation.

The composition of the invention may further comprise suitable matrixes in order to induce a controlled release of the EVs to the injured or diseases tissue, to stabilize the EVs and/or to enhance their therapeutic effect.

Suitable matrixes to be used in the present invention are capable of encapsulating the EVs and release them in a controlled manner, either in case of injection or cutaneous application, or are capable of acting as an inert carrier of bioactive molecules. Suitable matrixes include, but are not limited to, scaffolds, films, hydrogels, hydrocolloids, membranes, foams, nanofibers, gels and sponges. To facilitate EV-matrix delivery, the formulation can be combined with medical devices, such as patches, surgical threads, gauzes.

In general, the compositions of the present invention formulated for topic application or local injection are particularly useful to promote tissue repair, wherein tissue is affected by impaired angiogenesis, or is exposed to bacterial infection. The invention provides the applicability of plant-derived extracellular vesicles as a therapeutic topic treatment promoting a therapeutic effect on damaged tissues and cellular repair, e.g. when the damaged tissues show impaired angiogenesis, or are exposed to microbial infections.

Compositions according to the present invention, comprising either native or loaded plant-derived EVs, wherein the EVs have pro-angiogenic activities, are particularly useful for therapeutic treatment of ulcers, such as pressure ulcers, arterial ulcers, venous ulcers, ischemic ulcers, diabetic ulcers, exudative ulcers, dysmetabolic ulcers, burns, fistulae, fissures, and cutaneous diseases, including psoriasis, dermatitis, acne, eczema, seborrheic dermatitis, atopic dermatitis, contact dermatitis, dyshidrotic eczema, neurodermatitis, dermatitis herpetiformis, keratosis, keratitis, corneal damages/eye diseases (including ulcers, traumatic injuries, degeneration injuries, abrasions, chemical injuries, contact lens problems, ultraviolet injuries, keratitis), dry eye, conjunctivitis, androgenic alopecia, pruritus.

Compositions according to the present invention, comprising either native or loaded plant-derived EVs, wherein the EVs have anti-bacterial activity, are particularly useful for the treatment of mucosal lesions (such as traumatic lesions due to prosthesis and such, diabetic, mouth, decubital, genital mucosal lesions), infective lesions (such as virus infections, herpes infections, bacterial infections), ulcers (including diabetic, arterial, venous, dysmetabolic, exudative, ischemic, pressure), burns, fistulae, fissures, corneal damages and eye diseases (including ulcers, traumatic injuries, degenerative injuries, abrasions injuries, chemical injuries, ultraviolet injuries, keratitis), dry eye, conjunctivitis, dermatitis (including acne, eczema, seborrheic dermatitis, atopic dermatitis, contact dermatitis, dyshidrotic eczema, neurodermatitis, dermatitis herpetiformis), cellular damage induced by pro-apoptotic drugs aimed to treat pre-cancerous lesions (e.g. actinic keratosis).

The dose of the pharmaceutical composition of the present invention may vary depending on various factors, including the activity of a particular compound used, the patient's age, body weight, general health, sex, diet, administration time, the route of administration, excretion rate, drug combination, and the severity of a particular disease to be prevented or treated, and can be suitably determined by a person skilled in the art depending on the patient's condition, body weight, the severity of the disease, the form of drug, the route of administration, and the period of administration.

A pharmaceutical composition according to the present invention may be formulated as pills, sugar-coated tablets, capsules, liquids, gels, syrups, slurries, or suspensions.

Pharmaceutical compositions according to the present invention formulated for local delivery are efficient for enhancing tissue regeneration and cellular repair. This delivery system guarantees a local efficient and time-controlled release of EVs to the site of the lesion. Moreover, the delivery system can also guarantee the stabilization and storage of EV preparation. Such pharmaceutical compositions for local delivery of native or loaded EVs can contain hydrocolloidal/hydrogel-matrixes suitable for the site and kind of lesion to be treated. The formulation is intended to enhance cellular and/or tissue repair. Matrix-containing compositions can be adjusted to meet the requirements of the lesion of interest (presence of exudate, burn, dry lesion, mucosal ulcer, suture). The matrix itself can also support the therapeutic effect of EVs and wrap and enhance EVs stability. Matrixes can be solid/gelatin or liquid at room temperature and preferably include hydrocolloidal/hydrogel-matrixes. Matrixes can be created with several compounds (or their chemical modifications) and/or their combination, and include, but are not limited to chitosan, gelatin, hydroxyapatite, collagen, cellulose, hyaluronic acid, fibrin, alginate, cyclodextrin, starch, dextran, agarose, chondroitin sulfate, pullulan, protamine, pectin, glycerophosphate and heparin synthetic polymers such as poly(ethylene glycol) (PEG), poly(glycolic acid) (PGA), poly(vinyl alcohol) [PVA], polycaprolactone [PCL], poly(D,L-lactic acid) (PDLLA), poly(N-isopropylacrylamide) [PNIPAAm] and copolymers such as poly(D,L-lactic-co-glycolic acid) (PDLLGA). These molecules can be used in their native or chemically-modified form. Such components may be used individually or in combination.

Additionally, pharmaceutical compositions of the invention formulated for local delivery of native or modified EVs can contain suitable excipients, preservatives, solvents or diluents according to conventional method. Excipients, preservatives, solvents or diluents include, but are not limited to, lactose, agar, dextrose, sucrose, glycol, sorbitol, triclosan, benzyl alcohol, mannitol, propyleneglycol, xylitol, erythritol, maltitol, starch, parabens, gum acacia, alginate, gelatin, calcium phosphate, calcium silicate, cellulose, methylcellulose, salicylic acid, microcrystalline cellulose, sorbic acid, creolin, polyvinylpyrrolidone, quaternary ammonium cations, citric acid, acetic acid, ascorbic acid, boric acid, algenic acid, methylhydroxy benzoate, glycerol, propylhydroxy benzoate, zinc pyrithione, talc, sulfites, magnesium stearate, benzoic acid, mineral oils, propionic acid, chlorobutanol, fillers, extenders, binders, wetting agents, disintegrants, surfactants, propylene glycol, polyethylene glycol, plant oils such as olive oil, ethyl oleate, witepsol, Macrogol, Tween, cocoa butter, laurin fat, glycerogelatin, purified water, oils, waxes, fatty acids, fatty acid alcohols, fatty acid esters, surfactants, humectants, thickeners, antioxidants, viscosity stabilizers, chelating agents, buffers, lower alcohols, vitamins, UV blocking agents, fragrances, dyes, antibiotics, antibacterial agents, or antifungal agents, and the like. These molecules can be used in their native form or with chemical modifications. Such components may be used individually or in combination.

The native or loaded plant-derived population of EVs, combined or not with matrixes, can be also used as active compounds in a food supplement preparation suitable as edible dietary supplement. The therapeutic properties of EVs can support cell renewal in the gastrointestinal tract.

Accordingly, the invention also encompasses an edible preparation containing native or loaded plant-derived EVs, preferably derived from Brassicaceae family, such as Anastatica hierochuntica; from Selaginella lepidophylla; from Asteraceae family, such as Calendula officinalis; from Oleaceae family, such as Olea europaea; from Xanthorrhoeaceae family, such as Aloe vera, from Nelumbonaceae family, such as Nelumbo; from Araliaceae family, such as Subgenus Panax; from Lamiaceae family, such as Lavandula; from Hypericaceae family, such as Hypericum perforatum; from Pedaliaceae family, such as Harpagophytum procumbens; from Ginkgoaceae family, such as Ginkgo biloba; Piperaceae family, such as Piper kadsura or Piper futokadsura; Rubiaceae family, such as Hedyotis diffusa.

The food supplement preparation may be formulated in several oral administrable forms, including powders, granules, tablets, capsules, suspensions, emulsions, syrups, aerosols. In addition, the dietary supplement of the present invention may contain various nutrients, vitamins, minerals (electrolytes), flavorings such as synthetic flavorings and natural flavorings, colorants, pectic acid and its salt, alginic acid and its salt, organic acids, protective colloidal thickeners, pH adjusting agents, stabilizers, preservatives, glycerin, alcohol, carbonizing agents as used in carbonated beverages, etc. Such components may be used individually or in combination.

The food supplement preparation of the invention may further contain suitable excipients, preservatives, solvents or diluents known to the skilled in the art. Excipients, preservatives, solvents or diluents include, but are not limited to, lactose, agar, dextrose, sucrose, glycol, sorbitol, triclosan, benzyl alcohol, mannitol, propyleneglycol, xylitol, erythritol, maltitol, starch, parabens, gum acacia, alginate, gelatin, calcium phosphate, calcium silicate, cellulose, methylcellulose, salicylic acid, microcrystalline cellulose, sorbic acid, creolin, polyvinylpyrrolidone, quaternary ammonium cations, citric acid, acetic acid, ascorbic acid, boric acid, algenic acid, methylhydroxy benzoate, glycerol, propylhydroxy benzoate, zinc pyrithione, talc, sulfites, magnesium stearate, benzoic acid, mineral oils, propionic acid, chlorobutanol, fillers, extenders, binders, wetting agents, disintegrants, surfactants, propylene glycol, polyethylene glycol, plant oils such as olive oil, ethyl oleate, witepsol, Macrogol, Tween, cocoa butter, laurin fat, glycerogelatin, purified water, oils, waxes, fatty acids, fatty acid alcohols, fatty acid esters, surfactants, humectants, thickeners, antioxidants, viscosity stabilizers, chelating agents, buffers, lower alcohols, vitamins, UV blocking agents, fragrances, dyes, antibiotics, antibacterial agents, or antifungal agents and the like. These molecules can be used in native form or with chemical modifications. Such components may be used individually or in combination.

### EXAMPLES

The following experimental section is provided purely by way of illustration and is not intended to limit the scope of the invention as defined in the appended claims. In the following experimental section, reference is made to the appended drawings, wherein:

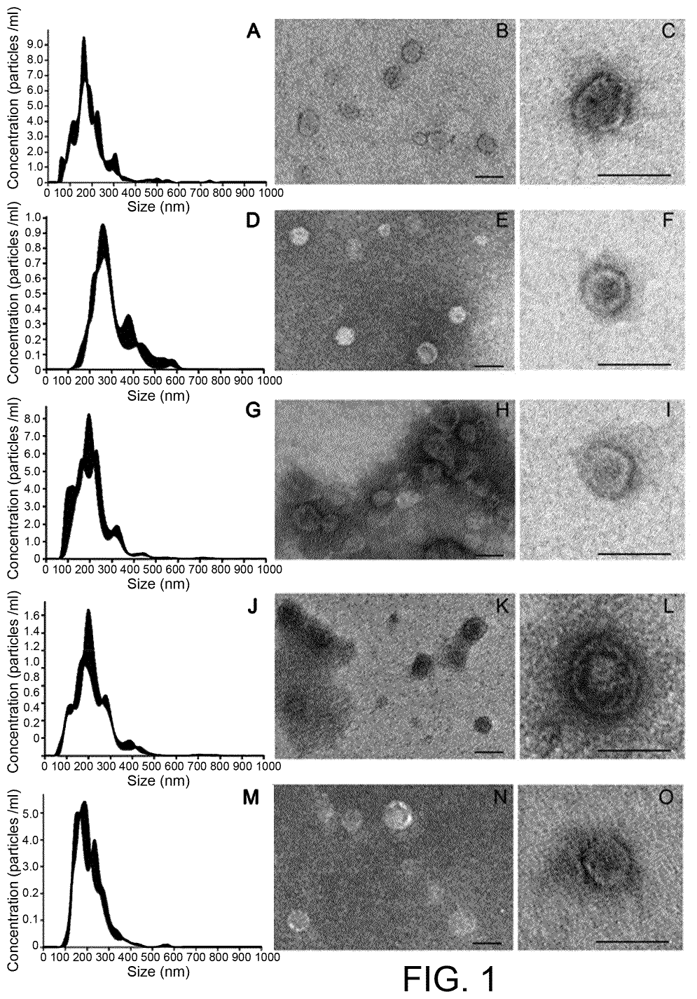

FIG. 1 shows the characterization of native plant-derived EVs in experimental example 1 for EVs derived from A) Lemon, B) Orange, C) Grape, D) Anastatica hierochuntica and E) Selaginella lepidophylla. Representative image of Nanosight analysis and transmission electron microscopy photographs of EVs (Original magnifications: ×40,000 and ×120,000) showed a size typical of EVs.

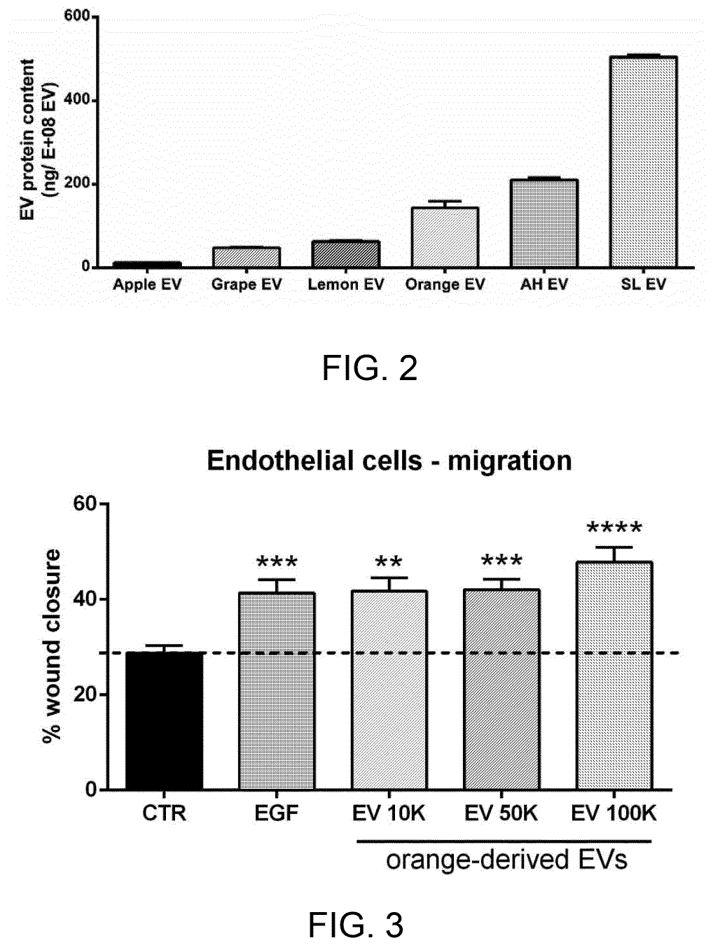

FIG. 2 shows the protein content of native plant-derived EVs in experimental example 1 expressed as nanograms (ng) of protein in 10⁸ EVs isolated from Apple, Lemon, Orange, Grape, Anastatica hierochuntica and Selaginella lepidophylla.

FIG. 3 shows the results of the promotion of endothelial cell migration in vitro mediated by native plant-derived EVs in experimental example 2. The graph shows the percentage of wound closure (mean±SEM) compared to control cells (CTR) measured by scratch test. Cells were treated with three different doses of native orange-derived EVs: 10,000 EVs/cell (EV 10 k), 50,000 EVs/cell (EV 50 k), 100,000 EVs/cell (EV 100 k). N=4 experiments were performed for each data set and Endothelial Growth Factor (EGF) 10 μM was used as positive control. The statistical significance was calculated comparing each condition with CTR. p: *<0.05; **<0.01; ***<0.005; ****<0.001.

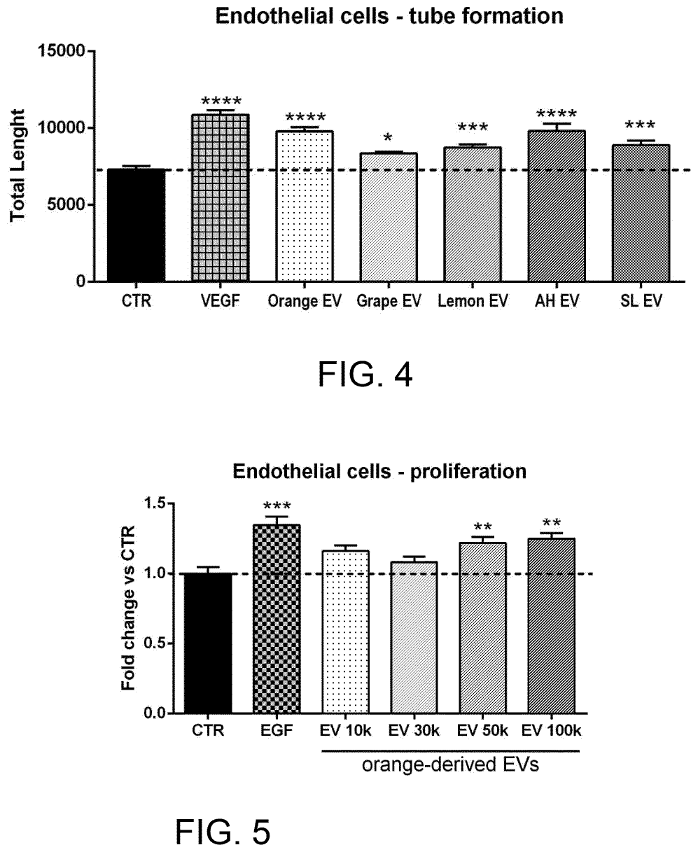

FIG. 4 shows the results of the ability of native plant-derived EVs to promote angiogenesis in experimental example 2. Endothelial cells were stimulated with EVs derived from Lemon, Orange, Grape, Anastatica hierochuntica (AH) and Selaginella lepidophylla (SL) (100,000 EVs/cell) and tube formation assay was performed. N=4 experiments were performed for each data set and Vascular Endothelial Growth Factor (VEGF) 10 μM was used as positive control. The statistical significance was calculated comparing each condition with CTR. p: *<0.05; **<0.01; ***<0.005; ****<0.001.

FIG. 5 shows the results of the native plant-derived EVs promotion of cell proliferation on hypoxia-stimulated endothelial cells in experimental example 2. Endothelial cells were incubated in hypoxic condition for 24 h and then treated with three different doses of orange-derived EVs (10,000 (10 k) or 30,000 (30 k) or 50,000 (50 k) or 100,000 (100 k) EVs/cell) for additional 24 h. Proliferation was tested by BrdU incorporation and analysis was performed comparing fold change versus control cells (CTR). EGF 10 μM was used as positive control (CTR+). The statistical significance was calculated comparing each condition with CTR. p: *<0.05; **<0.01; ***<0.005; ****<0.001.

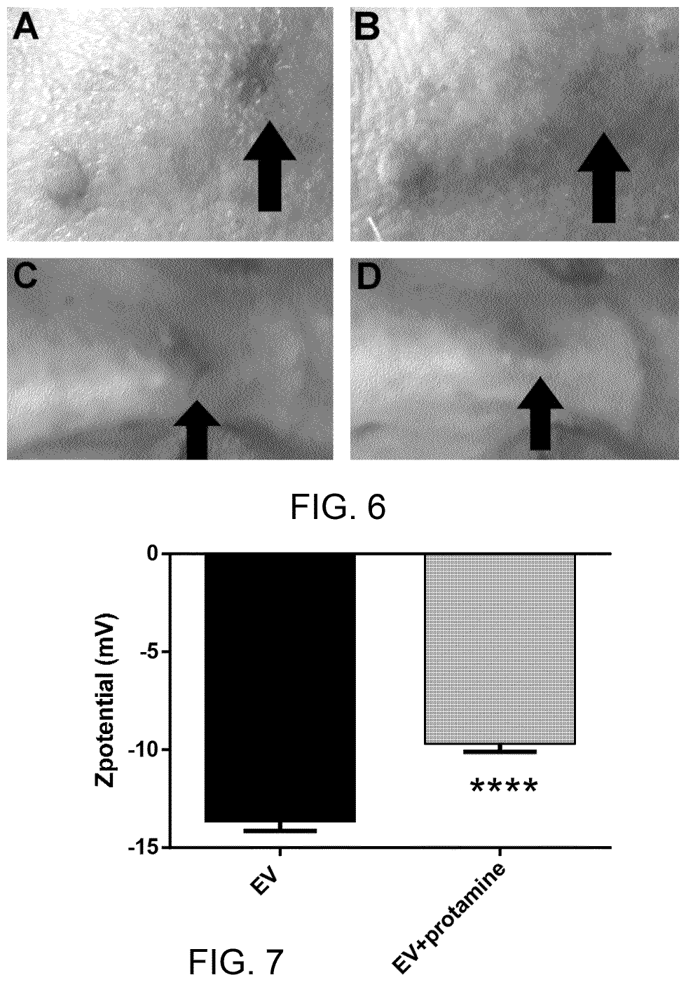

FIG. 6 shows the results of the in vivo therapeutic effects of native plant-derived EVs in human in experimental example 4. Native orange-derived EVs were used to treat a skin damage induced by Ingenol mebutate (ingenol-3-angelate, Picato) used for the topical treatment of a pre-cancerous lesion, the actinic keratosis. Representative images of tissue lesions were shown: the lesion before (FIG. 6A) and after (FIG. 6B) a treatment of three days with plant-derived EVs in comparison to untreated lesion before (FIG. 6C) and after (FIG. 6D) three days.

FIG. 7 shows the results of EV charge measurements in experimental example 5. Z-potential (mV), index of particle charge, was measured in native EVs (EV) derived from orange and EVs engineered with protamine 1.0 μg/ml (EV+protamine). Results derived from three experiments in triplicate. p: ****<0.001.

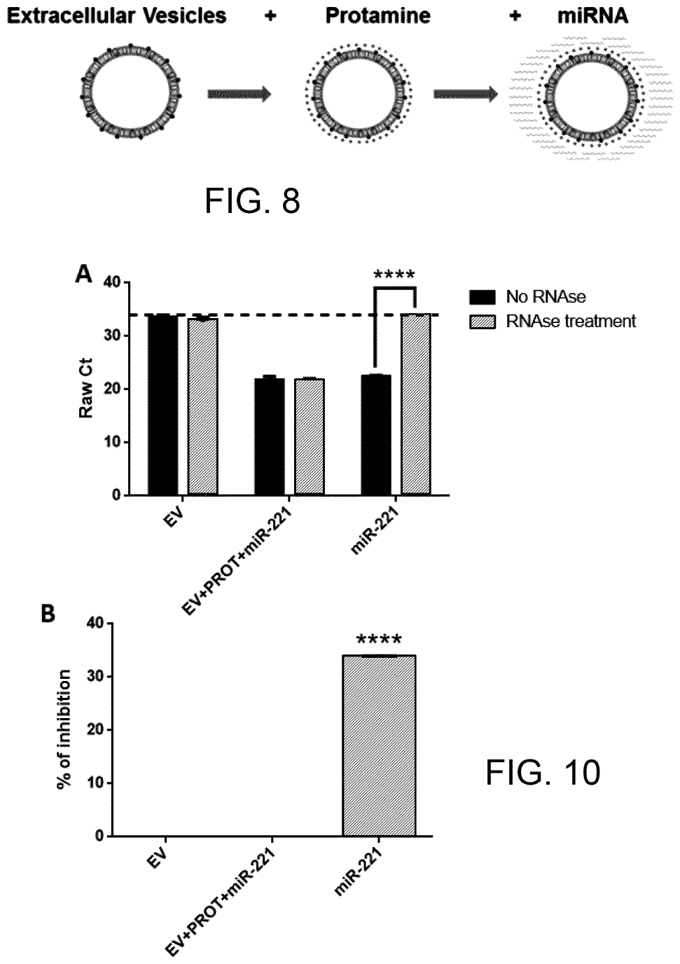

FIG. 8 illustrates the method of EV modification in experimental example 5. The invention consists in using a positive-charged molecule (like protamine) as a bridge for binding of negative-charged biologically active molecules (for instance miRNAs) to concentrate the molecules on EV surface.

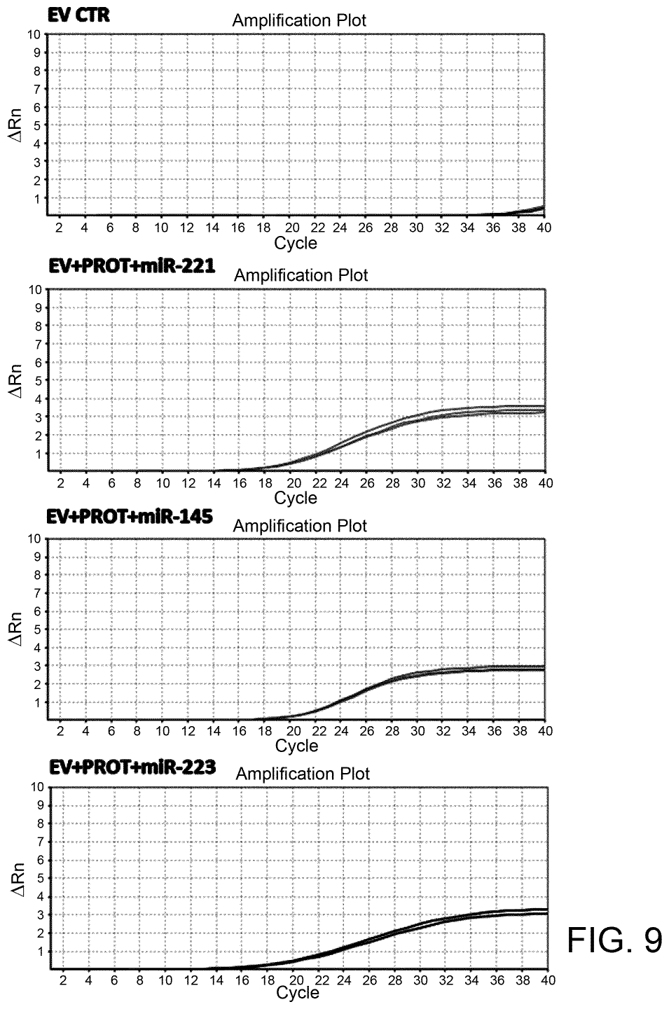

FIG. 9 shows the results of miRNA presence in loaded EVs in experimental example 5. Amplification plot obtained by qRT-PCR analysis of native orange-derived EVs (EV CTR), EVs engineered with protamine and synthetic human miRNA, miR-145, miR-221, or miR-223 (EV+PROT+miR-145/miR-221/miR-223). miRNA expression is represented as ΔRn, the magnitude of the signal derived from miRNA amplification, versus number of cycles.

FIG. 10 shows the results of the protection of engineered molecules (miRNA) after RNAse treatment in experimental example 5. Orange-derived EVs engineered with miRNA miR-221 were treated with a physiological concentration of RNAse (0.2 μg/ml) and the miRNA expression was evaluated by qRT-PCR in control native EVs (EV), loaded EVs as EVs engineered with protamine and miRNA (EV+PROT+miR-221), and free miRNA (miR-221). Data are reported as Raw Ct (A) and percentage of inhibition in respect to not treated samples (B). p:****<0.001.

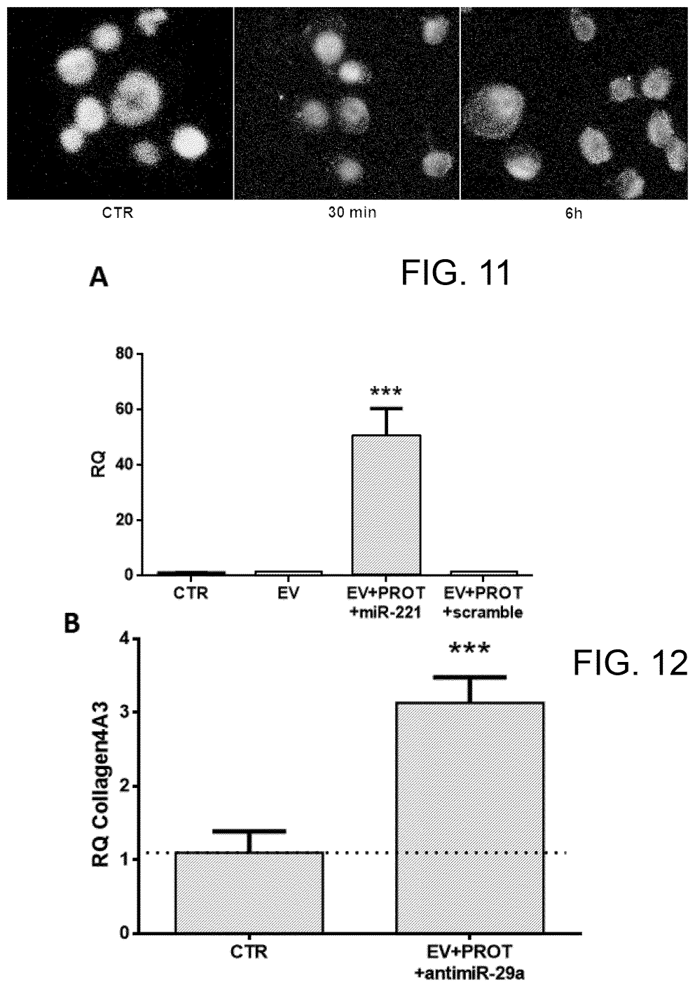

FIG. 11 shows the EVs incorporation in target cells using confocal microscopy in experimental example 6. Endothelial cells (TEC) were treated with fluorescent labeled loaded orange-derived EVs (30,000 EVs/cells) for different timing (30 min. 6 hours) and analyzed by confocal microscopy to detect their entrance in target cells. Representative micrograph of cells treated with stained EVs (EV CTR), or with labeled EVs for 30 minutes and 6 hours are shown. EV membrane, miRNA, cell nuclei were stained with red-PKH26, green-FITC, blue-DAPI, respectively. (Original magnification: ×630)

FIG. 12 shows the direct transfer of loaded miRNA in target cells and its functionality in experimental example 6. Endothelial cells (TEC) were cultured with normal medium (CTR), native orange-derived EVs (EV), loaded orange-derived EVs engineered with protamine and miRNA miR-221 (EV+PROT+mimic-221) or scramble miRNA (EV+PROT+scramble) or antimiR-29a (EV+PROT+antimir-29a) (30,000 EVs/cell). A) The transfer of miRNA (miR-221) in target cells was evaluated by qPT-PCR analysis using RNU6B as miRNA housekeeping and cells cultured without stimuli as control. The data are presented as RQ values and compared to CTR. B) Effect on Collagen4A3 mRNA target after treatment with EVs engineered with miRNA (antimir-29a). Evaluation of miRNA activity on its target mRNA Collagen4 isoform A3 after 72 hours. Cells were co-incubated with loaded EVs engineered with antimiR-29a (EV+PROT+antimir-29a) (30,000 EVs/cell) or normal medium (CTR) and the expression of Collagen4A3 was evaluated by qRT-PCR. The data are presented as RQ values. The data are presented as RQ values and compared to CTR. p: ***<0.005.

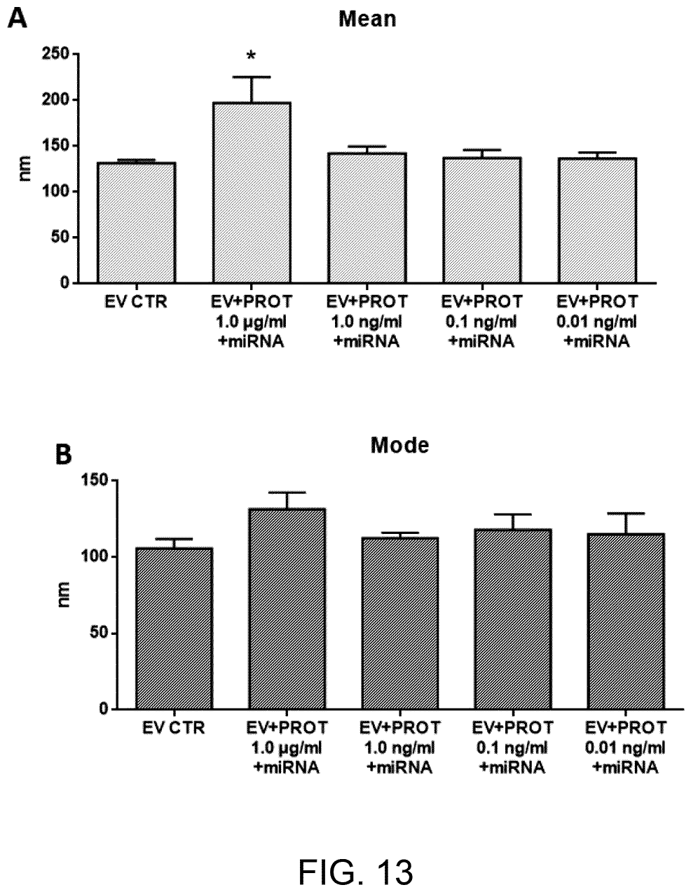

FIG. 13 shows the size analysis of loaded EVs engineered with decreasing doses of protamine in experimental example 7. Nanosight analysis of control native orange-derived EVs (EV CTR), loaded EVs engineered with protamine (initial dose, 1.0 μg/ml) and lower doses: 1.0 ng/ml, 0.1 ng/ml, 0.01 ng/ml. After co-incubation with miRNA (miR-221), EV analysis was evaluated as mean A) mode B) size of loaded EVs. The data were compared to EV CTR (native EVs). p: *<0.05.

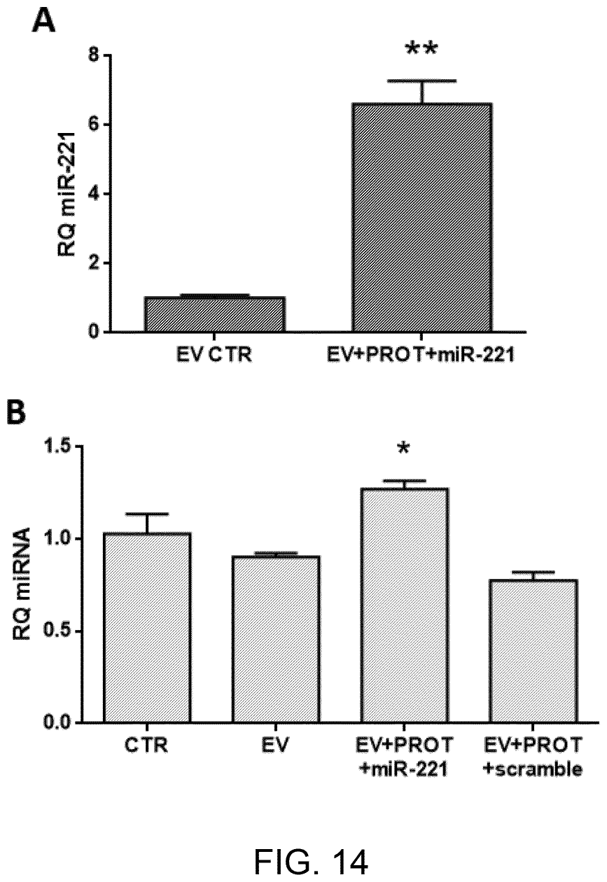

FIG. 14 shows the results of the miRNA expression in loaded EVs after engineering and its incorporation in target cells using a lower dose of protamine in experimental example 7. A) Loaded orange-derived EVs engineered with the lower dose of protamine (1.0 ng/ml) and miRNA miR-221 and analyzed for their content of exogenous miRNA. Data, obtained by qRT-PCR analysis, are shown as RQ values, using RNU6B as housekeeping gene and normalized with native EVs (EV CTR). p: **<0.01. B) Loaded orange-derived EVs engineered with protamine (1.0 ng/ml) and miRNA miR-221 or scramble, and co-incubated with endothelial cells (TEC) for 24 hours. The presence of loaded miRNA was measured in target cells by qRT-PCR and presented as RQ in cells cultured with normal medium (CTR), normal native EVs (EV), or loaded EVs engineered using protamine and miRNA scramble (EV+PROT+scramble) or miR-221 (EV+PROT+miR-221). p: *<0.05.

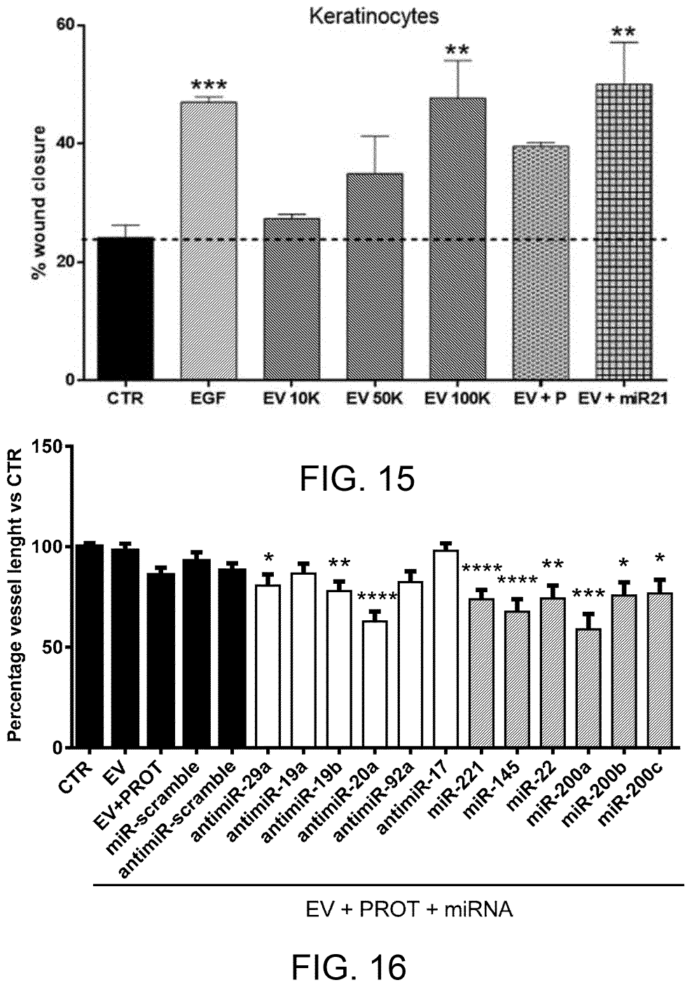

FIG. 15 shows the improvement of the therapeutic effect of native plant-derived EVs following the engineering with pro-regenerative miRNAs in experimental example 8. The graph illustrates the enhanced migration of keratinocytes and shows the percentage of wound closure (mean±SEM) compared to control cells (CTR). Cells were treated with three different doses of native orange-derived EVs: 10,000 EV/cell (EV 10 k), 50,000 EV/cell (EV 50 k), 100,000 EV/cell (EV 100 k); and a dose of 5,000 EV/cell of loaded EVs plus protamine (1.0 ng/ml) (EV+P) and loaded EVs plus protamine and miR-21 (EV+miR-21). EGF (10 μM) was used as positive control. N=4 experiments were performed for each data set. The statistical significance was calculated comparing each condition with CTR.

FIG. 16 shows the acquisition of new biological functions by loaded EVs following engineering with miRNAs in experimental example 9. Loaded orange-derived EVs engineered with several antiangiogenic miRNAs were tested on vessel formation of endothelial cells (TEC) using angiogenesis assay. TECs were cultured with normal medium (CTR), native EVs (EV), loaded EVs engineered with protamine (EV+protamine), or loaded EVs modified with protamine (1.0 ng/ml) and a synthetic antiangiogenic miRNA (antimiR for proangiogenic miRNAs and miR for antiangiogenic miRNAs). Scrambles are control miRNAs. After 24 hours of treatment, the total length of vessels was measured, and the percentage of total length is reported compared to normal cells (CTR). p: *<0.05, **<0.01, ***<0.005, ****<0.001.

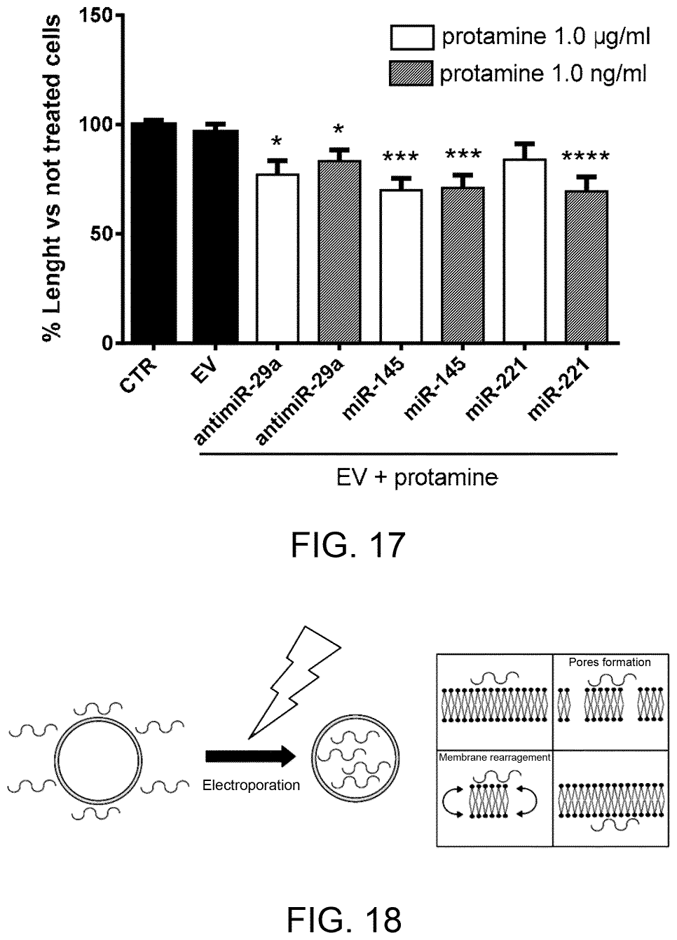

FIG. 17 shows the results of the biological activity of loaded EVs engineered with two different doses of protamine in experimental example 10. Orange-derived EVs were engineered with the initial (1.0 μg/ml) or a lower (1.0 ng/ml) amount of protamine and different antiangiogenic miRNAs (antimiR-29a, miR-145, miR-221). Loaded EVs were used to treat endothelial cells (TEC) and the vessel formation was evaluated in comparison to control cells (CTR) and cells cultured with native EV (EV). Total length is reported as percentage in respect to control cells p: *<0.05, ***<0.005, ****<0.001.

FIG. 18 illustrates the enhancement of molecule internalization using the modification method described in the present patent and the addition of a common transfection method in experimental example 11. Binding of a negatively-charged molecule (such as miRNA) to EV increases the number of molecules on EV surface and increases their loading after a transfection protocol, such as electroporation. In fact, the elevated number of molecules on EV surface allows an enhanced loading inside EVs following the membrane rearrangement that favors the flip of miRNA inside EVs.

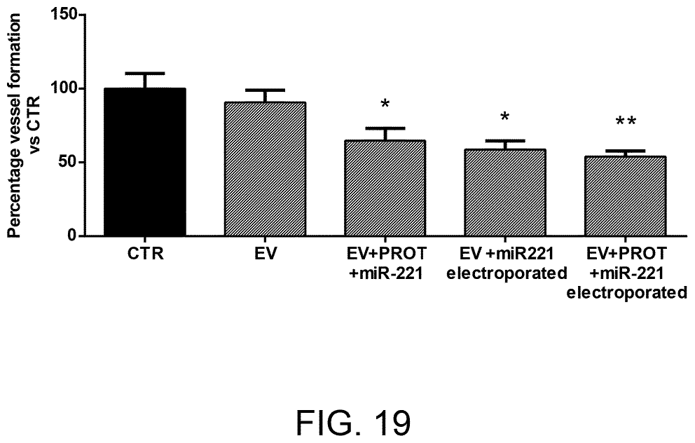

FIG. 19 shows the results of the enhancement of engineering using a combination of the modification method described in the present patent and the addition of a common transfection method in experimental example 11. Endothelial cells (TEC) were stimulated for 24 hours and the vessel formation was measured using angiogenesis assay. Stimuli were normal medium (CTR), native orange-derived EVs (EV), loaded EVs engineered using protamine (1.0 ng/ml) and miRNA miR-221 (EV+PROT+miR-221), EVs electroporated with miR-221 (EV+miR-221 electroporated), and loaded EVs electroporated after modification with protamine (1.0 ng/ml) and miRNA miR-221 (EV+PROT-miR-221 electroporated). Vessel formation was evaluated as percentage of vessel formation compared to normal cells (CTR). p: *<0.05, **<0.01.

### MATERIALS AND METHODS

### Extracellular Vesicles Isolation

Extracellular vesicles were isolated from plant juice. Fruits were squeezed and the juice was sequentially filtered using decreasing order of pores to remove fibers. EVs were then purified with ultracentrifugation. For differential ultracentrifugation the juice was first centrifuged at 1,500 g for 30 minutes to remove debris and other contaminants. Then, EVs were purified by a first centrifugation at 10,000 g followed by ultracentrifugation at 100,000 g for 1 hour at 4° C. (Beckman Coulter Optima L-90K, Fullerton, Calif., USA). The final pellet was resuspended with phosphate buffered saline added with 1% DMSO and filtered with 0.22 micrometer filters to sterilize. Extracellular vesicles were used or stored at −80° C. for long time. Purified EVs were characterized by nanoparticle tracking analysis and electron microscopy.

### Nanoparticle Tracking Analysis (NTA)

Nanoparticle tracking analysis (NTA) was used to define the EV dimension and profile using the NanoSight LM10 system (NanoSight Ltd., Amesbury, UK), equipped with a 405 nm laser and with the NTA 3.1 analytic software. The Brownian movements of EVs present in the sample subjected to a laser light source were recorded by a camera and converted into size and concentration parameters by NTA through the Stokes-Einstein equation. Camera levels were for all the acquisition at 16 and for each sample, five videos of 30 s duration were recorded. Briefly, purified EVs and engineered EVs were diluted (1:1000 and 1:200, respectively) in 1 ml vesicle-free saline solution (Fresenius Kabi, Runcorn, UK). NTA post-acquisition settings were optimized and maintained constant among all samples, and each video was then analyzed to measure EV mean, mode and concentration.

### Transmission Electron Microscopy

Transmission electron microscopy of EVs was performed by loading EVs onto 200 mesh nickel formvar carbon coated grids (Electron Microscopy Science, Hatfield, Pa.) for 20 min. EVs were then fixed with a solution containing 2.5% glutaraldehyde and 2% sucrose and after repeated washings in distilled water, samples were negatively stained with NanoVan (Nanoprobes, Yaphank, NK, USA) and examined by Jeol JEM 1010 electron microscope.

### Cell Culture

Human microvascular endothelial cells (HMEC) were obtained by immortalization with simian virus 40 of primary human dermal microvascular endothelial cells. HMEC were cultured in Endothelial Basal Medium supplemented with bullet kit (EBM, Lonza, Basel, Switzerland) and 1 ml Mycozap CL (Lonza, Basel, Switzerland).

Immortalized human keratinocytes (HaCat) were cultured with DMEM (Lonza, Basel, Switzerland) supplemented with 10% Fetal Bovine Serum (FBS, Thermo Fisher Scientific, Waltham, Mass., USA) at 37° C. with 5% CO2. The cells were seeded at density 3.5×102 cell/cm2, using 1 ml of medium/cm2 and subcultured when cell confluence was 70-80%. Briefly, flasks were washed with HEPES buffer saline solution, incubated with trypsin solution for 6 min and then trypsin was neutralized with medium containing 10% FBS. If the cells were not completely detached within 7 min, incubation with trypsin was repeated.

Endothelial cells derived from human renal carcinoma (TECs) were isolated from specimens of clear-cell type renal cell carcinomas using anti-CD105 Ab coupled to magnetic beads by magnetic cell sorting using the MACS system (Miltenyi Biotec, Auburn, Calif., USA). TEC cell lines were established and maintained in culture in Endogro basal complete medium (Merck Millipore, Billerica, Mass., USA). TEC were previously characterized as endothelial cells by morphology, positive staining for vWF antigen, CD105, CD146, and vascular endothelial-cadherin and negative staining for cytokeratin and desmin.

### Protein Analysis

Proteins were extracted from EVs by RIPA buffer (150 nM NaCl, 20 nM Tris-HCl, 0.1% sodium dodecyl sulfate, 1% deoxycholate, 1% Triton X-100, pH 7.8) supplemented with a cocktail of protease and phosphatase inhibitors (Sigma-Aldrich, St. Louis, Mo., USA). The protein content was quantified by BCA Protein Assay Kit (Thermo Fisher Scientific, Waltham, Mass., USA) following manufacturer's protocol. Briefly, 10 μl of sample were dispensed into wells of a 96-well plate and total protein concentrations were determined using a linear standard curve established with bovine serum albumin (BSA).

### In Vitro Scratch-Test Assay

Keratinocyes (HaCaT) and endothelial cells (HMEC) were seeded at a density of ˜50×10³ cells/well in 24-well plates in DMEM supplemented with 10% FCS. When the cells reached complete confluence, they were starved with medium without FCS overnight. The following day, scratch wounds were created with a sterile tip. Prior to stimulation (t=0), micrographs of the well were obtained using a Leica microscope (Leica, Wetzlar, Germany). The cells were then stimulated with DMEM with 10% FBS or EGF as positive controls (CTR+) or EVs (10,000 (10 k) or 50,000 (50 k) or 100,000 (100 k) EVs/target cells). The ‘wound closure’ phenomenon was monitored for 48 hr using the Leica microscope and images were analyzed by ImageJ software (Bethesda, Md., USA) observing the decrease of the wound area in cells stimulated with EVs in comparison to cells not stimulated with EVs.

### In Vitro Angiogenic Assay

In vitro formation of capillary-like structures was studied on growth factor-reduced Matrigel (BD Bioscience, Franklin Lakes, N.J., USA) in 24-well plates. HMEC or TECs (25,000 cells/well) were seeded onto Matrigel-coated wells in DMEM or EndoGRO MV-VEGF medium, respectively, containing EVs (50,000 or 30,000 EVs/target cells). Treatments were performed in triplicate. Cell organization onto Matrigel was imaged with a Nikon Eclipse TE200. After incubation for 24 h, phase-contrast images (magnification, ×10) were recorded and the total length of the network structures was measured using ImageJ software. The total length per field was calculated in five random fields and expressed as a percentage to the respective control.

### In Vitro Proliferation Assay

HMEC were plated in a 96 well plate at a density of 2,000 cells/well and left to adhere.

The culture medium was replaced with DMEM to leave overnight. Then, the plate was closed in a hypoxic chamber filled with the following mixture of gas: 5% CO2, 1% 02, 94% N. The hypoxic chamber was placed in CO2 incubator for 24 h. Then the plate was removed from hypoxic chamber, cells were treated with DMEM alone (CTR), positive control (10 ng/ml of EGF, CTR+), increasing doses of native plant derived EVs (10,000, 30,000, 50,000, and 100,000 EVs/cell). Each condition is performed in quadruplicate. Then 10 μl of BrdU labeling solution (BrdU colorimetric assay, Roche) were added to each well and the plate was incubated overnight. The following procedure was performed according with BrdU assay manufacturer's instruction. Absorbance was measured by an ELISA reader at 370 nm. The mean absorbance for each condition was calculated. Absorbance is directly proportional to proliferation rate. All mean absorbances were normalized for the mean of untreated control (CTR), used as reference samples. The results show the relative proliferation rate compared to CTR, which is equal to 1.

### Measurement of EV Charge

The analysis was performed by Zeta-sizer nanoinstrument (Malvern Instruments, Malvern, UK). All samples were analyzed at 25° C. in filtered (cutoff=200 nm) saline solution. Zeta-potential (slipping plane) is generated at x distance from the particle indicating the degree of electrostatic repulsion between adjacent, similarly charged particles in a dispersion. Negative Zeta-potential indicates a high grade of dispersion across the particles.

Engineering of EVs with Protamine

EVs were mixed with protamine (1.0 μg/ml) (Sigma-Aldrich, St. Louis, Mo.) and co-incubated at 37° C. for 5-30 minutes to allow the binding to EV surface. Various doses of protamine (1.0 ng/ml, 0.1 ng/ml, 0.01 ng/ml) was used. Next, synthetic miRNA molecules (100 pmol/ml) (miRNA mimics or antimiR, Qiagen, Hilden, Germany), negative-charged, were added to the mixture and co-incubated at 37° C. for 3 hours. The mixture was diluted with saline solution and stored at 4° C. overnight. Ultracentrifugation with 100,000 g for 2 hours at 4° C. (Beckman Coulter Optima L-90K, Fullerton, Calif., USA) allowed the elimination of free miRNA and protamine molecules, and the pellet was resuspended with phosphate buffered saline added with 1% DMSO and filtered with 0.22 micrometer filters to sterilize.

### RNAse Treatment

EVs were treated with RNAse A (Thermo Fisher Scientific, Waltham, Mass., USA), using a concentration of 0.2 μg/ml, for 30 minutes at 37° C. The RNAse inhibitor (Thermo Fisher Scientific, Waltham, Mass., USA) was used to stop the reaction as described by the manufacturer's protocol and EVs were washed by ultracentrifugation at 100,000 g for 1 hour at 4° C. (Beckman Coulter Optima L-90K, Fullerton, Calif., USA).

### Confocal Microscopy

For EV incorporation, EVs were labeled with a red membrane fluorescent dye for membranes, PKH26 (Sigma-Aldrich, St. Louis, Mo.) and engineered with a green fluorescent (FITC) labeled siRNA (Qiagen, Hilden, Germany). Labeled-EVs were used to treat TEC plated in 24-well plates (30,000 cells/well) for different timing (30 min, 1 h, 3 h, 6 h, 18 h, 24 h). The uptake of EVs was analyzed using confocal microscopy (Zeiss LSM 5 Pascal, Carl Zeiss, Oberkochen, Germany).

### RNA Extraction

Total RNA was isolated from EVs and cells using the miRNeasy Mini Kit (Qiagen, Hilden, Germany) according to the manufacturer's protocol. RNA concentration of samples was quantified using spectrophotometer (mySPEC, VWR, Radnor, Pa., USA).

MiRNA and mRNA Analysis by qRT-PCR

For miRNA analysis, miScript SYBR Green PCR Kit (Qiagen, Hilden, Germany) was used. Briefly, RNA samples were reverse transcribed using the miScript Reverse Transcription Kit and the cDNA was then used to detect and quantify miRNAs of interest. Experiments were run in triplicate using 3 ng of cDNA for each reaction as described by the manufacturer's protocol (Qiagen). For mRNA analysis, cDNA was obtained using High-Capacity cDNA Reverse Transcription Kit (Applied Biosystems). Five nanograms of cDNA were added to SYBR GREEN PCR Master Mix (Applied Biosystems) and run on a 96-well QuantStudio 12K Flex Real-Time PCR (qRT-PCR) system (Thermo Fisher Scientific, Waltham, Mass., USA). GAPDH was used as a housekeeping gene. Fold change (Rq) in miRNA expression among all samples was calculated as 2^{-ΔΔCt} compared to control samples.

### Extracellular Vesicle Electroporation

Electroporation was performed on a Neon Transfection System (Thermo Fisher Scientific, Waltham, Mass., USA) following manufacturer's protocol. For every electroporation, the sample volume was fixed at 200 μL.

### In Vivo Experiments

Ingenol mebutate (ingenol-3-angelate, Picato) was used for the topical treatment of pre-cancerous lesions induced by actinic keratosis. The drug was applicated for 3 days on actinic keratosis lesions removing the pre-cancerous lesion but inducing the formation of tissue apoptotic lesions. After the treatment, native orange-derived EVs were topically administered on one tissue lesion, whereas one untreated lesion on the same patient was used as control. The effect of plant-derived EVs was evaluated after 3-7 days of treatment.

### Statistical Analysis

Data analysis was carried out with the software package Graph Pad Demo version 6.01. Results are expressed as mean±standard error (SEM). One way analysis of variance (ANOVA) was used to substantiate statistical differences between groups, while Student's t-test was used for comparison between two samples. We used p<0.05 as a minimal level of significance. p: *<0.05, **<0.01, ***<0.005, ****<0.001.

### RESULTS/EXAMPLES

### Example 1

To investigate the feasibility of the method of the present invention, the inventors used native EVs purified from different plants, including lemon, orange, grape, Anastatica hierochuntica and Selaginella lepidophylla. EVs were isolated by microfiltration and differential ultracentrifugation or tangential flow filtration and they displayed a size in the range of 25-350 nm by Nanosight analysis (FIG. 1). Moreover, all native plant-derived EVs showed a round morphology delimited by an electrondense membrane as demonstrated by electron microscopy analysis (FIG. 1).

In order to examine the content of plant-derived EVs, the protein content of native EVs isolated from apple, orange, lemon, grape, Anastatica hierochuntica and Selaginella lepidophylla was measured by using the BCA assay. The results are shown in the FIG. 2 and demonstrate a heterogeneous protein content for EVs illustrated in Table 1.

TABLE 1
EV protein content.

| | mean protein ng/E+08 | |
|---|---|---|
| | EV | SD |
| Apple EV | 12.06 | 0.71 |
| Grape EV | 47.93 | 2.17 |
| Lemon EV | 63.13 | 4.55 |
| Orange EV | 143.74 | 49.21 |
| AH EV | 210.58 | 11.05 |
| SL EV | 504.58 | 13.06 |

Moreover, deeper analysis demonstrated that native plant-derived EVs contain proteins characteristic of vesicle, such as HSP70, HSP80, glyceraldehyde-3-phosphate dehydrogenase (G3PD) and fructose-bisphosphate aldolase 6 (FBA6); and plant proteins, such as Patellin-3-like and clathrin heavy chain.

In addition, the lipid content of native plant-derived EVs revealed a cargo of lipids variable in amount depending on the plant, including 24-Propylidene cholesterol, Beta sitosterol, Glycidol stearate, Dipalmitin, Campesterol, Eicosanol, Eicosane, Hexadecane, Hexadecanol, Octadecane, Octadecanol, Tetradecane, Tetradecene, Valencene and Stearate.

### Example 2

The ability of native plant-derived EVs to promote endothelial cell migration and angiogenesis was tested. By performing a scratch on a monolayer of endothelial cells, the present inventors observed a significantly higher migration rate of endothelial cells using different doses of native plant-derived EVs compared to negative control (CTR) (FIG. 3), demonstrating that plant-derived EVs can promote migration of endothelial cells and support angiogenesis.

In addition, the capacity of native plant-derived EVs to promote vessel formation was evaluated by angiogenesis assay to verify their effect on vessel formation in vitro. Results showed in FIG. 4 demonstrated that all native plant-derived EVs tested significantly promoted the formation of new vessels by stimulating endothelial cells, thus promoting angiogenesis.

Moreover, the ability of native plant-derived EVs to promote cell proliferation on endothelial cells was tested after hypoxic damage in vitro. Different doses of native orange-derived EVs significantly promoted cell proliferation rate compared to negative control (CTR), demonstrating the beneficial effect of EVs on endothelial cells (FIG. 5).

### Example 3

Native plant-derived EVs were analyzed for their anti-microbial activity. Most of the pathogenic bacteria associated with infected lesions in humans need a pH value>6 and their growth is inhibited by lower pH values. Native plant-derived EVs show a low pH ranging from 4 to 5. Applying native plant-derived EVs to the lesion surface creates an acidic environment unfavorable for the growth and the multiplication of bacterial pathogens, such as Pseudomonas aeruginosa, Staphylococcus aureus, Escherichia coli, Klebsiella spp., Proteus spp., Citrobacter spp., S. epidermidis, S. pyogenes, streptococci, and enterococci.

The application of plant-derived EVs is effective in clearing bacterial pathogens from contaminated or infected lesions by lowering the pH. In fact, the treatment with native plant-derived EVs restored the average surface pH of the skin (normally ranging from about 4.2 to 5.6) controlling the topic infection increasing the natural antimicrobial activity of the skin. Moreover, the decrease of pH has been demonstrated to enhances the antibacterial activity of other drugs against both gram-positive and gram-negative bacteria.

### Example 4

Native plant-derived EVs were analyzed for their therapeutic effect in vivo. Native plant-derived EVs were used to treat a skin damage induced by Ingenol mebutate (ingenol-3-angelate, Picato) in a human volunteer. This substance is an inducer of cell death and was used for the topical treatment of a pre-cancerous lesion, the actinic keratosis. Results illustrated in FIG. 6 shows the lesion before (FIG. 6A) and after (FIG. 6B) a treatment of three days with native plant-derived EVs in comparison to untreated lesion that was similar before (FIG. 6C) and after (FIG. 6D) three days. Native plant-derived EVs showed a therapeutic effect in a pro-apoptotic lesion induced by ingenol mebutate, promoting tissue regeneration after three days in comparison to untreated lesion.

### Example 5

In order to modify plant-derived EVs with a method based on charge interaction, native plant-derived EVs were analyzed for their surface charge. The analysis of Zeta potential was performed on different preparations showing that native EVs derived from orange display a negative charge of −13.59±1.83 mV (FIG. 7). Other native plant-derived EVs showed similar negative charge. Interestingly, orange derived EVs co-incubated with a positive charged linker, protamine, and washed by ultracentrifugation demonstrated a significantly increase in their charge, suggesting a modification of their surface (FIG. 7).

To investigate the method to load negatively-charged molecules on EV surface using a charged positive linker, loaded plant-derived EVs modified using protamine were mixed with miRNA molecules as illustrated in FIG. 8. As an example, loaded orange derived EVs using protamine were mixed with different miRNA mimics (miR-145, miR-221, miR-223) and the analysis by qRT-PCR demonstrated a clear enrichment of miRNAs in loaded EVs in respect to control native EVs (FIG. 9), suggesting an efficient molecule binding to EVs. Of interest, miRNAs associated with EVs were protected from degradation by the physiologic concentration of RNase present in biological fluids thus conferring biologic stability. FIG. 10A shows the complete inactivation of free miRNA by RNase treatment, whereas the miRNA bound to EVs was protected from inactivation, in comparison to native EVs that not express the miRNA. The percentage of miRNA inhibition in all samples is showed in FIG. 10B.

### Example 6

In order to understand whether loaded molecules could be efficiently transferred to target cells, the transfer of loaded molecules mediated by loaded plant-derived EVs to target cells was analyzed. Firstly, orange derived EVs were labeled with PKH26 (red fluorescent dye) and engineered with FITC fluorescent labeled synthetic siRNA. Loaded EVs were co-incubated with human endothelial cells derived from renal carcinoma (TECs) at different time points (30 min, 1 h, 3 h, 6 h, 18 h, 24 h). Analysis by confocal microscopy revealed that small nucleic acids present on EV's surface did not alter their uptake by target cells. FIG. 11 shows control cells (CTR) labelled for nuclei and that the treatment with loaded EVs increases the fluorescent signal in target cells already after 30 minutes of co-incubation, with a greatest uptake at 6 hours (FIG. 11). In addition, the efficient transfer of loaded EVs was also demonstrated by the detection of the uptake by target cells. For this purpose, TECs were treated with loaded orange derived EVs modified with miRNA mimic-221 and analyzed by qRT-PCR after 24 h (dose 30,000 EVs/cell). As shown in the FIG. 12A, miRNAs were efficiently transferred into target cells through EVs. The functionality of loaded molecules in target cells was also tested. For this purpose, TEC cells were stimulated with loaded orange derived EVs engineered with anti-miR-29a and the expression of mRNA target gene was measured in target cells by qRT-PCR experiments. Results demonstrated that miRNAs transferred to target cells by EVs were also functional and induced a significantly increase of its target gene Collagen4A3 (FIG. 12B).

### Example 7

In order to deeper investigate the use of a positive charged linker, different doses of protamine were evaluated to load plant-derived EVs. Positively-charged molecules, such as protamine, can form micelles around negatively-charged molecules, such as miRNAs. Then, orange derived EVs were co-incubated with decreasing doses of protamine and a representative negatively-charged molecule, the miRNA miR-221-3p. The size analysis of EVs performed by Nanosight measured the mean and mode size of loaded EVs. Results showed that the initial amount of protamine (1.0 μg/ml) induced an increase in both mean and mode size, with a significantly difference in mean (FIG. 13). However, this alteration in EV size was not present when EV where co-incubated with lower doses of protamine (1.0 ng/ml, 0.1 ng/ml, 0.01 ng/ml). The results suggested an excess of protamine dose for the initial amount, resulting in the formation of micelles with a greater size than normal native EVs present in EV preparation. To verify that lower doses of positive charged linker were sufficient to allow an interaction with negative charged molecules, the expression of loaded miRNA molecules was measured in loaded plant-derived EVs and their transfer was evaluated in target cells. The qRT-PCR analysis of loaded orange derived EVs engineered with a representative miRNA (miR-221-3p) using a lower dose of protamine (1.0 ng/ml) demonstrated that miRNA was efficiently bound to EVs (FIG. 14a). Moreover, loaded orange derived EVs modified with a lower dose of protamine (1.0 ng/ml) were able to efficiently transfer miRNAs to target cells as demonstrated by qRT-PCR analysis of target cells treated with loaded EVs (FIG. 14b).

### Example 8

As a representative example of the modification method to further improve the native activity of plant-derived EVs, the modification of plant-derived EVs was used to improve their native activity in promoting wound closure of keratinocytes. In these experiments, loaded orange derived EVs were engineered with miRNA miR-21 using protamine as positively-charged linker. Human keratinocytes were treated with three different doses of native EVs, loaded EVs incubated with protamine alone (EV+P) as control, and loaded EVs with protamine and miR-21 (EV+miR-21). EV+P and EV+miR-21 was used at the intermediate dose (50 k). The measurement of the wound closure in each condition was used as parameter of EV activity. The graph in the FIG. 15 shows that EV+P promote wound closure as well as the same doses of native EVs, while EV+miR-21 promote a statistically significant increase in wound closure, as well as a double dose of native EVs (EV 100 k). The results demonstrated that the modification method can be used to increase the therapeutic effects of native plant-derived EVs.

### Example 9

The modification method can also be used to change the biological activity of native plant-derived EVs. Plant-derived EVs can be engineered with negatively-charged molecules that provide different or new biological effects. As a representative example, orange derived EVs were modified with different anti-angiogenic miRNAs and their ability to inhibit angiogenesis was evaluated by angiogenesis assay in vitro on TEC cells. In particular, loaded EVs were engineered with mimics for anti-angiogenic miRNAs (miR-221, miR-223, miR-145) and anti-miRNAs for pro-angiogenic miRNAs (miR-29, miR-126, miR-31) and their effect on endothelial cell vessel formation was evaluated after 24 hours of treatment (FIG. 16). The results demonstrated that loaded orange derived EVs engineered with anti-angiogenic molecules were able to significantly inhibit angiogenesis of endothelial cells in vitro.

### Example 10

The therapeutic effect of loaded plant-derived EVs was evaluated using different doses of a positively-charged linker. As a representative example, loaded orange derived EVs were engineered with two different doses of protamine (1 μg/ml, 1 ng/ml) and three different antiangiogenic miRNAs (antimir-29, miR-145 and miR-221-3p). Loaded EVs were used to stimulate endothelial cells (TEC) for 24 hours and their activity was evaluated by angiogenesis assay. The results showed in FIG. 17 demonstrated that the activity of loaded EVs engineered with a lower dose of protamine was equally or more effective, demonstrating the feasibility of different doses of a positively-charged linker to efficiently modify native plant-derived EVs.

### Example 11

The modification method of plant-derived EVs with negatively-charged molecules can be further improved by transfection protocols. In fact, the bound of negatively-charged molecules (e.g. miRNAs) to EV surface through a positively-charged linker (e.g. protamine) could facilitate their entrance inside EVs. The closeness of negatively-charged molecules to EVs could increase their loading after transfection protocols such as electroporation, as illustrated in FIG. 18. A positively-charged linker, by forming a bridge between the negatively-charged EVs and negatively-charged molecules, concentrate on EV surface the molecules and favor the flip inside (FIG. 18). Membrane rearrangement obtained with strategies other than electroporation, such as sonication, mechanical cell extrusion, saponin-mediated permeabilization, and freeze-thaw cycles, were also shown to implement EV loading.

To test this hypothesis, orange-derived EVs were modified using protamine and the miR-221-3p and their capacity to inhibit vessel formation was evaluated. The FIG. 19 shows that the use of a transfection protocol, such as electroporation, on loaded EVs was able to increase their effect and improve their inhibitory activity on vessel formation on endothelial cells.

1. A method of promoting angiogenesis and inhibiting bacterial growth in a subject in need thereof, said method comprising administering to said subject a composition comprising a population of plant-derived extracellular vesicles (EVs), the plant-derived extracellular vesicles (EVs) in said population being delimited by a lipid bilayer membrane and having a diameter ranging from 10 to 500 nm, a protein content in the range of from 1 to 55 ng/10⁹ EVs, and an RNA content in the range of from 10 to 60 ng/10¹⁰ EVs, wherein said subject is affected by a disease or condition selected from the group consisting of ulcers, dermatites, corneal damages, eye diseases, mucosal lesions and infective lesions.

2. The method of claim 1, wherein the ulcers are selected from the group consisting of pressure ulcers, arterial ulcers, venous ulcers, diabetic ulcers, ischemic ulcers, exudative ulcers, dysmetabolic ulcers, traumatic ulcers, burns, fistulae, fissures and traumatic ulcers.

3. The method of claim 1, wherein the dermatites are selected from the group consisting of acne, eczema, seborrheic dermatitis, atopic dermatitis, contact dermatitis, dyshidrotic eczema, neurodermatitis, dermatitis herpetiformis, keratosis, keratitis and psoriasis.

4. The method of claim 1, wherein the corneal damages and eye diseases are selected from the group consisting of ulcers, traumatic injuries, degeneration injuries, abrasions, chemical injuries, contact lens problems, ultraviolet injuries and/or keratitis, conjunctivitis and dry eye.

5. The method of claim 1, wherein the mucosal lesions are selected from the group consisting of traumatic lesions due to prosthesis, diabetic, mouth, decubital or genital mucosal lesions.

6. The method of claim 1, wherein the infective lesions are selected from the group consisting of virus infections, herpes infections and bacterial infections.

7. The method of claim 1, wherein the EVs have a diameter in the range of from 20 to 400 nm.

8. The method of claim 1, wherein the EVs population is derived from one or more plants selected from the group consisting of: the family Rutaceae, the family Rosaceae, the family Vitaceae, the family Brassicaceae, the family Selaginellaceae, the family Asteraceae, the family Oleaceae, the family Xanthorrhoeaceae, the family Nelumbonaceae, the family Araliaceae, the family Lamiaceae, the family Hypericaceae, the family Pedaliaceae, the family Ginkgoaceae, the family Piperaceae, and the family Rubiaceae.

9. The method of claim 8, wherein the EVs population is derived from one or more plants selected from the group consisting of: the genus Citrus, including lemon, orange, tangerine, clementine, bergamot, pompia; Malus pumila; Vitis vinifera; Anastatica hierochuntica; Selaginella lepidophylla; Calendula officinalis; Olea europaea; Aloe vera; Nelumbo; the subgenus Panax; Lavandula; Hypericum perforatum; Harpagophytum procumbens; Ginkgo biloba; Piper kadsura, Piper futokadsura; and Hedyotis diffusa.

10. The method of claim 9, wherein the EVs are native EVs or EVs loaded with one or more negatively-charged biologically-active molecules selected from the group consisting of drugs, nucleic acid molecules and lipophilic molecules, including lipophilic vitamins, wherein the nucleic acid molecules are selected from the group consisting of miRNA, mRNA, tRNA, rRNA, siRNA, regulating RNA, non-coding and coding RNA, DNA fragments and DNA plasmids.

11. (canceled)

12. The method of claim 1, wherein the composition is formulated as a pharmaceutical composition for topic application, local injection or oral administration, or is formulated as a food supplement preparation.

13. A method for loading one or more negatively-charged biologically active molecules into a population of plant-derived extracellular vesicles (EVs), comprising the steps of:
(i) contacting and co-incubating a population of plant-derived extracellular vesicle (EVs) as defined in claim 1, with a polycationic substance and one or more negatively-charged biologically active molecules; and
(ii) purifying the loaded EVs obtained in step (i) from the polycationic substance and the remaining one or more free negatively-charged active molecules.

14. The method of 13, wherein the polycationic substance is selected from the group consisting of protamine, polylisine, cationic dextrans and combinations thereof.

15. The method of claim 13, wherein the one or more negatively-charged biologically active molecules are selected from the group consisting of drugs, nucleic acid molecules and lipophilic molecules, including lipophilic vitamins, wherein the nucleic acid molecules comprise miRNA, mRNA, tRNA, rRNA, siRNA, regulating RNA, non-coding and coding RNA, DNA fragments and DNA plasmids.
